(19) United States
(12) Plant Patent Application Publication
(10) Pub. No.: US 2013/0191953 P1
(43) Pub. Date: Jul. 25, 2013

(54) Sansevieria Plant Named 'Whitney'

(50) ;

(71) Applicant: Marvin Gonzalez Villegas, San Ramon (CR)

(72) Inventor: Marvin Gonzalez Villegas, San Ramon (CR)

(73) Assignee: FLORICA FARMS, INC.

(21) Appl. No.: 13/374,903

(22) Filed: Jan. 23, 2012

(51) Int. Cl. A01H 5/00

(52) U.S. Cl. PLT/382

(57) ABSTRACT

The Whitney variety belongs to the “compact Sansevieria group”, with a growth rate of 45-50 cm, and an average of 4 leaves per plant. The plant shape is an inverted pyramid and the arrangement of the leaves is in the form of a rosette. Present dark green color in the center of the blades with wide and variegated margins with irregular light green and dark green thin bands.

### LATIN NAME OF THE GENUS AND SPECIES OF THE CLAIMED PLANT

Sansevieria trifasciata

### VARIETY DENOMINATION

WHITNEY

### BACKGROUND OF THE INVENTION

The present invention relates to a new and distinct cultivar of Sansevieria plant, botanically known as Sansevieria trifasciata, of the family Ruscaceae, hereinafter referred to by the cultivar name “WHITNEY”

Sansevieria is a monocotyledonous and succulent, flowering plant which can be produced in either hard-leafed or soft-leafed varieties for interior use as a house plant or outdoor ornamental plant.

The new Sansevieria ‘WHITNEY’ originated from a naturally-occurring mutation of the Sansevieria variety ‘Black Gold Superba’ (unpatented). The new Sansevieria ‘WHITNEY’ was discovered and selected by the inventor, Marvin Gonzalez Villegas, as a single flowering plant within the progeny of the stated mutation in a controlled environment in 2006 in Monterrey, San Carlos, Alajuela province, Costa Rica.

Asexual reproduction of the new Sansevieria cultivar by vegetative cuttings was first performed in January 2007 in Monterrey, San Carlos, Alajuela, Costa Rica and has demonstrated that the combination of characteristics as herein disclosed for the new cultivar are firmly fixed and retained through successive generations of asexual reproduction. The new cultivar reproduces true to type.

### BRIEF SUMMARY OF THE INVENTION

The following traits have been repeatedly observed and are determined to be unique characteristics of ‘WHITNEY’, which in combination distinguish this Sansevieria as a new and distinct cultivar:

1. Dark green-colored foliage with variegated dark green and pale green margins, no stripes;

2. Wide, long (34 cm), lanceolate leaves supported in a rosette.

3. 21-40 cm plant height.

4. Erect plant habit.

5. Needs little water and is very drought tolerant.

The new variety WHITNEY is considered a variety of Sansevieria in the group denominated as “compact Sansevieria plants”. The claimed variety presents an average of 4-5 leaves per plant when it reaches its maturity (between 7-8 months). The claimed variety has an erect growth habit and the entire plant can reach a height range between 40 and 50 cm. The leaves are narrow (6.0 cm average measured at the widest part of the mature leaf) and lanceolate, with a soft waxy texture and the blades are thin and slightly wavy. The plant has an inverted triangle shape.

This variety is moderately drought tolerant and adapts to conditions of light and shade (interior and exterior).

The leaf blade is dark green (RHS 139A), and has 1.5 cm margins on both sides of the leaf. These margins are variegated with irregular horizontal stripes alternating with pale green (RHS 130D) wider stripes in young leaves. This color becomes lime green (RHS 142D) in mature leaves. In the fully mature leaves the base displays a bright yellow-green color (RHS 150A). These stripes alternate with thinner and weaker dark green ones (RHS 139A).

Plants of the new Sansevieria ‘WHITNEY’ differ from plants of the parental cultivar, ‘Black Gold Superba’ (unpatented) in the characteristics described in Table 1.

The variety ‘Black Gold Superba’ gave rise to the var. Whitney. The ‘Black Gold Superba’ variety is also considered to be part of the “compact Sansevieria group”. ‘Black Gold Superba’ shows less vigor than Whitney, has fewer leaves per plant (3-4), with an average leaf width of 6.5 cm (measured in the middle of the mature leaf) The growth habit is erect and presents wide leaves supported in a rosette. The entire plant can reach a size between 30-40 cm, and the plant has, as for Whitney, an inverted triangle shape. The leaves are lanceolate with a soft, waxy texture. The younger leaves show a yellowish-green vivid coloration (RHS 134A) in the interior of the leaf blade. This area also has irregular streaks of yellowish-green bands (RHS 142D), and there are margins on both sides of the leaf which are variegated with irregular horizontal bands that alternate between light yellowish green (RHS142D) broad bands and brilliant yellowish green (RHS 149A) thin bands. When the leaf is mature the deep color inside turns yellowish green (RHS 141B), while margins alternate between vivid greenish-yellow (RHS 2A) and light yellowish-green (RHS 2C) colors.

TABLE 1
Comparison with Parental Varieties

| | | Female Parent |
|---|---|---|
| | New Cultivar | Black Gold Superba |
| Characteristic | Whitney | (unpatented) |
| Plant Vigor | more vigorous (more | less vigorous (less |
| | leaves) | leaves) |
| Color | variegated leaves | green leaves with yellow |
| | (medium green cross- | margins. |
| | banding and dark | |
| | green margins) | |
| Shape | wide and lanceolate | wide and lanceolate |
| | leaves on a rosette | leaves |
| Texture of | smooth-textured leaves | smooth-textured leaves |
| leaves | | |

Of the many commercial cultivars known to the present inventor, the most similar in comparison to the new Sansevieria ‘WHITNEY’ is the Sansevieria trifasciata var. ‘Black Robusta’ (unpatented), in the characteristics described in Table 2

‘Black Robusta’ is described as a variety with a medium vigor, has a growth rate between 16-40 cm, and a quantity of 3-5 leaves per plant. The leaves are wide (8 cm in the widest part of the mature leaf) and lanceolate. The leaf maintains the same coloration in both upper and under sides. It has a uniform dark green color (RHS 139A) with stripes distributed throughout, with brilliant yellowish-green color (RHS 142B). The plant has an inverted triangle shape. As with other varieties of Sansevieria, this variety shows a high adaptability to conditions of light and shade, as well as tolerance to different water stress conditions.

TABLE 2
Comparison with Comparison Cultivar

| | | Comparison Cultivar |
|---|---|---|
| | New Cultivar | ‘BLACK ROBUSTA’ |
| Characteristic | ‘WHITNEY’ | (unpatented) |
| Plant Vigor | more vigor (4-5 leaves per | less vigor (3-4 leaves per plant) |
| | plant) | similar size |
| Color | dark green foliage with | dark green foliage, with pale |
| | variegated margins (dark | green irregulars stripes. |
| | green and pale green) | |
| Shape | wide and lanceolate leaves | wide and lanceolate leaves on a |
| | on a rosette | rosette |
| Texture of | smooth and waxy textured | smooth and waxy textured leaves |
| leaves leaves | leaves | |

### BRIEF DESCRIPTION OF THE PHOTOGRAPHS

The accompanying photographs illustrate the overall appearance of the new Sansevieria cultivar ‘WHITNEY’ showing the colors as true as is reasonably possible with colored reproductions of this type. Colors in the photographs may differ slightly from the color value cited in the detailed botanical description which accurately describe the color of ‘WHITNEY’.

FIG. 1 Shows a side view of an eight month old plant of Sansevieria ‘Whitney’ in a 21 cm diameter pot.

FIG. 2 shows a top view of an eight month old plant of Sansevieria ‘Whitney’ in a 21 cm diameter pot.

FIG. 3A Shows a close-up view of the upper side of a typical leaf of ‘Whitney’, taken from an eight-month old plant.

FIG. 3B Shows a close-up view of the lower side of a typical leaf of ‘Whitney’, taken from an eight-month old plant.

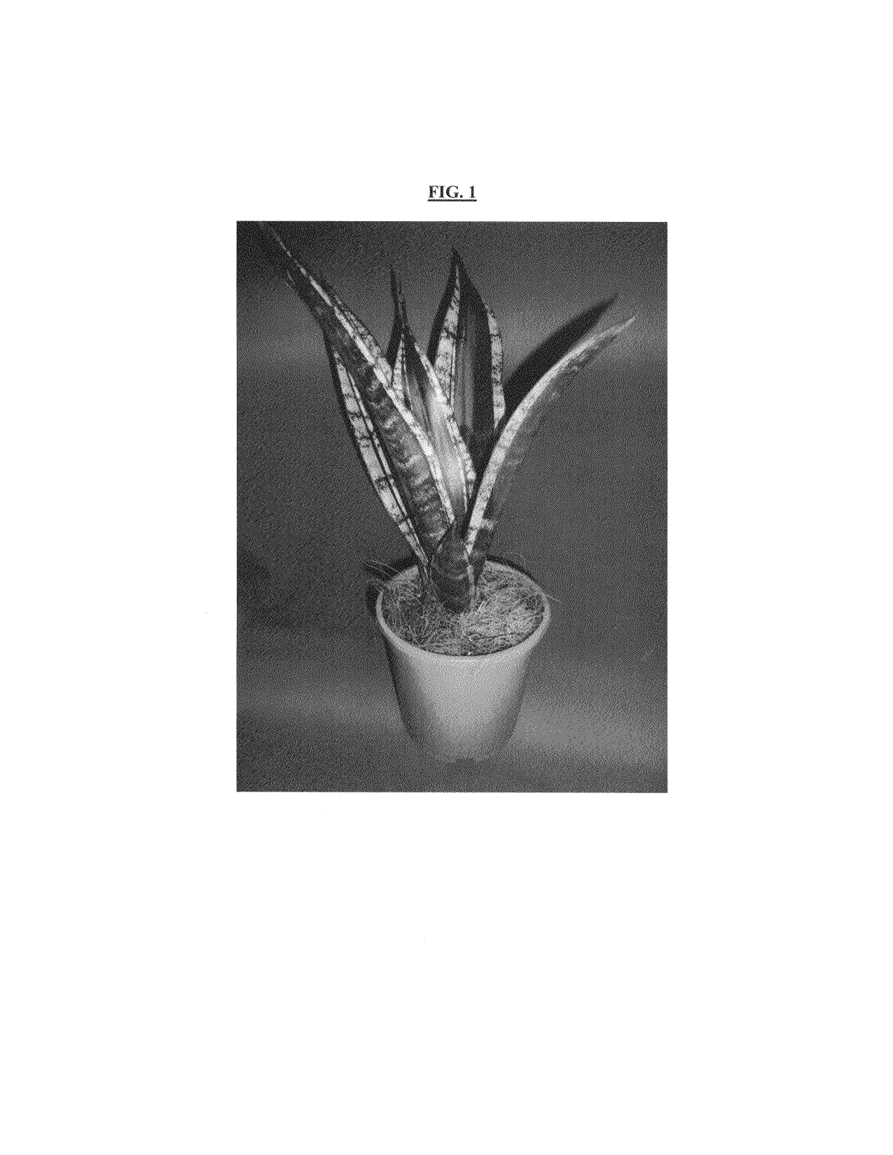

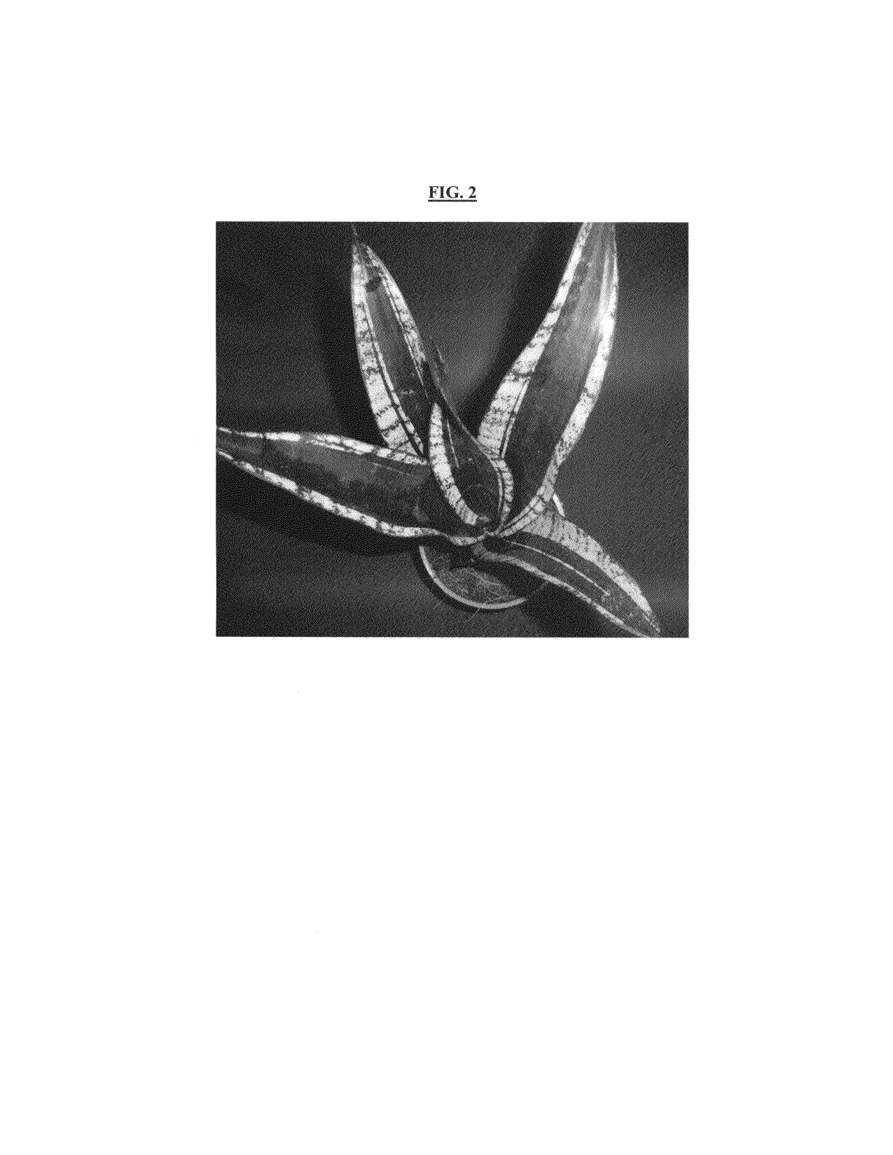

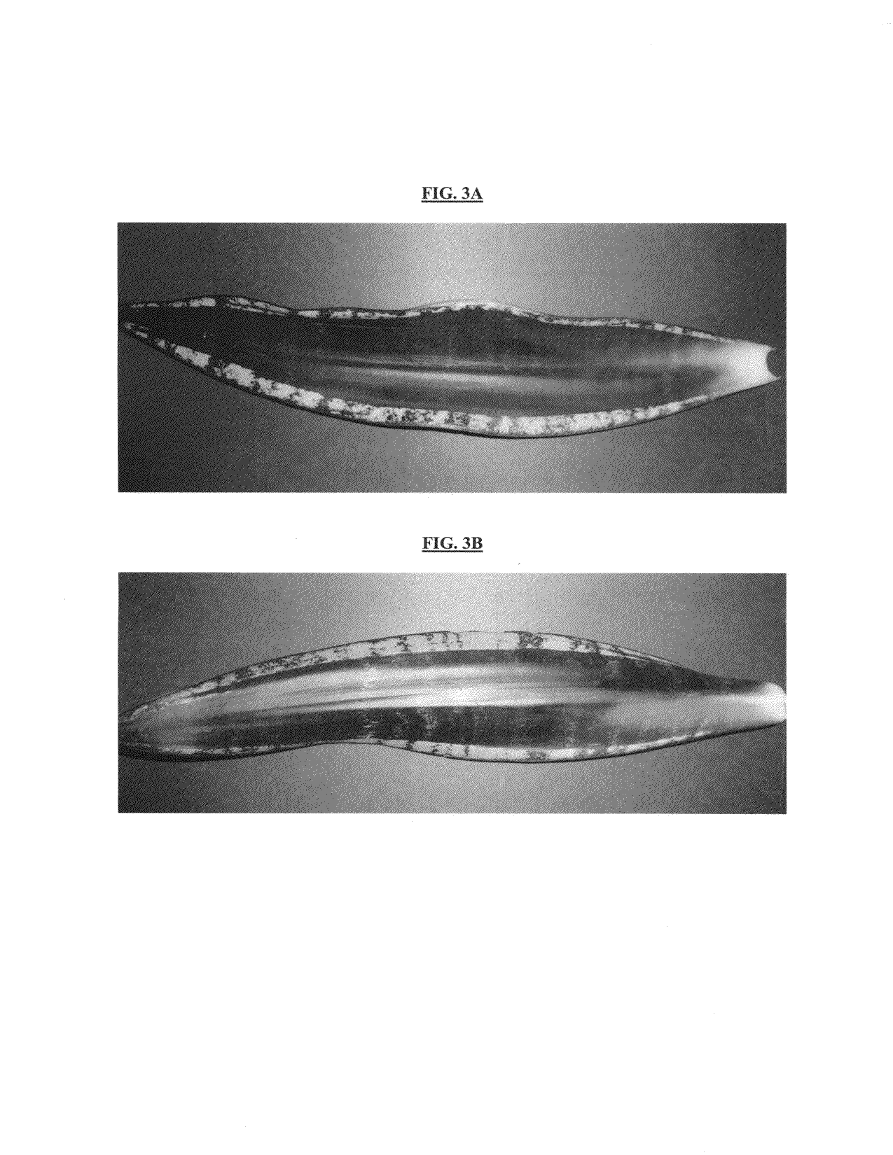

### DETAILED BOTANICAL DESCRIPTION

The new Sansevieria ‘WHITNEY’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe the new Sansevieria cultivar ‘WHITNEY’ as grown in open-field at Monterrey, San Carlos de Alajuela province, Costa Rica, conditions which closely approximate those generally used in commercial practice. Average temperature in Monterrey is 24.5° C. during the day and 18° C. at night. Plants grow under natural light conditions.

This crop does not require constant fertilization, however to increase production per area about 200 kg ha⁻¹ N, 150 kg ha⁻¹ of P and 100 kg ha⁻¹ K are required. Production is closely related with soil conditions in which the plants are grown.

Color references are made to The Royal Horticultural Society Color Chart (R.H.S.), (April 2009) except where general colors of ordinary significance are used.

The photographs and descriptions were taken during the rainy season in Monterrey, San Carlos, Alajuela province, Costa Rica when outdoor day temperature was 23° C. The age of the plants described is 8 months.
Classification:
   Botanical.—Sansevieria trifasciata.
   Variety denomination.—Whitney.
Parentage: Sansevieria trifasciata, ‘Black Gold Superba’ (unpatented)
Optimal growth conditions:
   Light intensities.—High adaptability to outdoor or indoor conditions.
   Temperature.—Day: 24° C. to 32° C. Night: 18° C. to 23° C.
   Temperature tolerance.—Tolerant to a low temperature of about 12° C. and tolerant to a high temperature of about 35° C.
   Fertilization.—200, 150 and 100 kg ha⁻¹ of N, P and K.
   Growth regulators.—Not necessary.
Propagation:
   Type.—Vegetative, by rooted cuttings.
   Rooting habit and description.—Rhizomes, with short roots.
   Time to initiate roots.—15 to 22 days at 18-25° C.
   Time to produce a rooted cutting.—About 60 to 90 days at 18-25° C.
Plant:
   General appearance and form.—
   Height.—About 41 cm when grown in ground; About 37 cm when grown in a 21 cm size container.
   Spread.—About 11 cm when grown in ground; About 10 cm when grown in 21 cm size container.
   Form.—Monocot; leaf bases arranged in a rosette around growth point.
   Shape.—inverted triangle.
   Growth rate and habit.—About 2 cm per week; upright.
   Fragrance.—None.
   Stem.—Modified (Rhizome and Foliage) Length: About 12 cm when grown in ground; About 5 cm when grown in 21 cm size container. Diameter: About 2 cm to 3.5 cm Shape: round. Texture: Rough Color: RHS 17A Strength: strong and durable Internode length: About 1 mm to 3 mm.
Foliage:
   Quantity.—About 5.
   Arrangement and attachment.—Single, alternate; leaf bases arranged in a rosette around growth point.
   Leaf length.—About 34 cm.
   Leaf width.—About 8.5 cm.
   Overall shape of leaf.—lanceolate.
   Apex shape.—acute, triangular.
   Base shape.—plane.
   Margin.—sharp, smooth.
   Texture.—Upper Surface: smooth and waxy texture Under Surface: smooth and waxy texture.
   Pubescence.—None.
   Color of mature leaf.—Upper Surface: RHS139A, Margins: irregular bands of RHS 150D and RHS139A. Lower Surface: RHS139A, Margins: irregular bands of RHS 150D and RHS139A.
   Color of immature leaf.—Upper Surface: RHS139A on the leaf surface, Margins: variegated with bands of RHS130D and RHS139A. Lower Surface: RHS 139A on the leaf surface, Margins: variegated with bands of RHS 130D and RHS139A.
   Venation.—Pattern: longitudinal/parallel leaf shape.
   Leaf fragrance.—None.
Inflorescence description:
   Arrangement and type.—Inflorescence.
   Orientation at opening.—out of the spike.
   Quantity.—Per Plant with at least one open flower: One inflorescence per plant About 22 flowers opened 2 weeks after appearance of the spike. Flowers sporadically and profusely under growth conditions.
   Inflorescence size.—Length: About 28 cm Diameter: About 0.4 mm.
   Flower size.—Length: About 2 cm Diameter: About 0.1 mm Depth: About 1 cm.
   Fragrance.—Soft and pleasant.
   Bud.—
   Rate of opening.—About 7 to 8 days, according to weather.
   Shape.—ovoid.
   Length.—About 22.3 cm.
   Diameter.—About 0.3 mm.
   Color.—Apex: RHS 150A Base: RHS 142A.
   Texture (both surfaces).—smooth.
Petals:
   Quantity.—6.
   Arrangement.—star-shaped.
Reproductive organs:
   Androecium.—Stamen: Number: 5 per flower. Length: About 1.3 cm Color: RHS 150C Anther: Length: About 0.2 mm Width: About 0.1 mm Color: RHS 150C Filament: Length: About 1.1 mm Color: RHS 150C Pollen: Amount: moderate Color: RHS 154D.
   Gynoecium.—Pistil: Number: One per flower Length: About 2.1 cm Stigma: Length: About 0.6 mm Width: About 0.8 mm Color: RHS 150C Style: Length: About 1.3 mm Color: RHS 150C Ovary: Length: About 3 mm Width: About 2.5 mm Color: RHS 150C.
Weather resistance: Drought tolerant
Disease/pest resistance: not observed for disease/pest resistance.
Disease/pest susceptibility: not observed for disease/pest susceptibility.

I claim:

1. A new and distinct variety of Sansevieria plant named ‘WHITNEY’, substantially as illustrated and described herein.
